(19) United States
(12) Patent Application Publication
(10) Pub. No.: US 2023/0137006 A1
(43) Pub. Date: May 4, 2023

(54) METHODS AND SYSTEMS FOR PRODUCING ELEMENTAL SELENIUM DURING SELENATE REMOVAL FROM WATER

(71) Applicant: ARIZONA BOARD OF REGENTS OF BEHALF OF ARIZONA STATE UNIVERSITY, Scottsdale, AZ (US)

(72) Inventors: Bruce Rittmann, Tempe, AZ (US); Chen Zhou, Chandler, AZ (US); Min Long, Tempe, AZ (US)

(21) Appl. No.: 17/916,795

(22) PCT Filed: Apr. 2, 2021
(86) PCT No.: PCT/US2021/025666

§ 371 Date: Oct. 3, 2022

Related U.S. Application Data
(60) Provisional application No. 63/005,955, filed on Apr. 6, 2020.

(51) Int. Cl. C02F 3/10; C02F 3/34

(52) U.S. Cl. CPC C02F 3/102; C02F 3/341; C02F 2101/10

(57) ABSTRACT

The present invention relates to systems and methods for removing selenate and/or selenite from water and recovering elemental selenium.

### CROSS-REFERENCE TO RELATED APPLICATIONS

This application claims the benefit of and priority to U.S. provisional patent application 63/005,955, filed Apr. 6, 2020, the entirety of the disclosure of which is hereby incorporated by this reference thereto.

### FIELD OF THE INVENTION

The invention relates to systems and methods for removing selenium contaminants and recovering elemental selenium with minimal over-reduction of selenium, for example from waste streams or wastewater.

### BACKGROUND OF THE INVENTION

Selenium (Se) can cause toxic effects on ecosystems and human health and is regulated by the USEPA and other agencies worldwide. Se's contamination in water bodies originates mainly from mining, mineral processing, and power generation. For example, the Se concentration in coal mine wastewaters can reach up to 10 mg/L. Though an essential trace element utilized by all living organisms, selenium can cause toxic effects on ecosystems and on human health due to its strong tendency to bioaccumulate and be biomagnified into organisms at higher levels of the food chain. As a result, Se-containing wastewaters must be treated before discharge. The U.S. Environmental Protection Agency (EPA) has set a maximum contamination level (MCL) for Se in drinking water as 50 μg/L. Environment and Climate Change Canada has set an ecotoxicity limit as low as 1 μg/L.

Se is present in a range of chemical forms. The dominant forms of Se in most water bodies are oxidized soluble oxyanions, hexavalent selenate (SeO₄²⁻) and tetravalent selenite (SeO₃²⁻). In principle, selenate and selenite can be removed from water through physiochemical processes such as adsorption onto metal oxides and ion exchange, but these processes generate significant amounts of hazardous wastes that significantly increase total treatment costs and also intensify environmental threats.

A reduction method can be utilized to transform these oxidized contaminants to the desirable reduced form, which is elemental selenium (Se⁰), a solid that has minimal toxicity and also is a valuable feedstock for the electronics industry. Over-reduction of selenium, however, generates undesired Se species, for example, Se at −2 oxidation state (also described herein as “Se(-II)”), namely, hydrogen selenide (H₂Se), Se⁻ and Se²⁻ anions, and organic selenium (organic-Se), which are serious ecological and human-health concerns. Thus, the challenge is finding a bioreduction process that reduces selenate contaminants without producing selenide or organic-Se.

### SUMMARY OF THE INVENTION

In one aspect, the disclosure relates to a method of generating a biofilm in a membrane biofilm reactor to maximize selenate and/or selenite removal from water and reduction to harmless, but valuable elemental selenium that is harvested in its most-valuable nanoparticle form. The method typically includes providing an aqueous system comprising a nonporous hollow-fiber membrane; inoculating the nonporous hollow-fiber membrane with hydrogenoautotrophic bacteria and contacting the aqueous system with hydrogen gas (H₂). The partial pressure of H₂ provided to the aqueous system is ±10% of the theoretical pressure of H₂ determined by combining the partial pressures of H₂ calculated from equation (4) and equation (6).

$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} \qquad ( 4 )$$

$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} \qquad ( 6 )$$

The inoculated aqueous system is then provided a first growth medium comprising nitrate to establish a biofilm on the nonporous hollow-fiber membrane; and then provided a second growth medium comprising selenium contaminants to enrich the biofilm for selenium-reducing bacteria. The enriched biofilm respires selenate and/or selenite to Se⁰ without producing selenide or organic-Se.

In some implementation, the inoculated aqueous system is cultured with the first growth medium for at least three weeks and cultured with the second growth medium for at least three weeks. In some aspects, the second growth medium lacks nitrate. In some aspects, the first growth medium contains only nitrate as an electron acceptor. In particular implementations, the concentration of nitrate in the first growth medium is 14-70 mg-N/L (1-4 mM) and the concentration of selenite in the second medium is 100-200 mg/L (0.7-1.4 mM) selenate.

In a second aspect, the disclosure relates to a method for removal of selenium contamination from a fluid while minimizing the generation of toxic selenide or organic-Se during the selenate bioreduction process. The method advantageously includes providing an aqueous system comprising a nonporous hollow-fiber membrane; inoculating the nonporous hollow-fiber membrane with hydrogenoautotrophic bacteria and contacting the aqueous system with hydrogen gas (H₂). The partial pressure of H₂ provided to the aqueous system is ±10% of the theoretical pressure of H₂ determined by combining the partial pressures of H₂ calculated from equation (4) and equation (6).

$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} \qquad ( 4 )$$

$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} \qquad ( 6 )$$

A first growth medium comprising nitrate to establish a biofilm on the nonporous hollow-fiber membrane is provided to the inoculated aqueous system. A second growth medium comprising selenium contaminants to enrich the biofilm for selenium-reducing bacteria is then provided, whereby a bioreactor for reducing selenate is produced. In some aspects, the enriched biofilm respires selenate and/or selenite to Se⁰ without producing selenide or organic-Se. The bioreactor for reducing selenate is then contacted with fluid containing selenium contaminant so that the biofilm enriched for selenium-reducing bacteria reduces selenium contaminate to Se⁰ and captures Se⁰ . In some embodiments, the method further comprises harvesting the biomass in the bioreactor to harvest Se⁰ generated by the bioreactor.

In certain implementation, the inoculated aqueous system is cultured with the first growth medium for at least three weeks and cultured with the second growth medium for at least three weeks. In some aspects, the second growth medium lacks nitrate. In some aspects, the first growth medium contains only nitrate as an electron acceptor. In particular implementations, the concentration of nitrate in the first growth medium is 14-70 mg-N/L (1-4 mM) and the concentration of selenite in the second medium is 100-200 mg/L (0.7-1.4 mM) selenate.

The disclosure also relates to a bioreactor system that enables controlled H₂ delivery through bubbleless gas-transfer membranes to a biofilm capable of selenate bioreduction without generating toxic selenide or organic-Se. The system for removing selenium contaminants and harvesting elemental selenium (Se⁰) from a fluid comprises a nonporous hollow-fiber membrane, an inoculant comprising hydrogenoautotrophic bacteria, and a hydrogen gas source. In some aspects, the hydrogen gas source provides H₂ at partial pressure of±10% of the theoretical pressure of H₂ determined by combining the partial pressures of H₂ calculated from equation (4) and equation (6).

$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} \qquad ( 4 )$$

$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} \qquad ( 6 )$$

In some embodiments, the hydrogen gas source comprises a hydrogen gas tank comprising H₂ gas and a gas pressure regulator, the gas pressure regulator regulates the flow of H₂ gas from the gas tank to the membrane.

In one embodiment, the system further comprises a growth medium, the growth medium comprising selenate. For example, the concentration of selenate in the second medium is 100-200 mg/L (0.7-1.4 mM).

In another embodiment, the system further comprises a first growth medium and a second growth medium, wherein the first growth medium comprises nitrate and the second growth medium comprises selenate. In some aspects, the second growth medium lacks nitrate. In some aspects, the first growth medium contains only nitrate as an electron donor. In certain embodiments, the concentration of nitrate in the first growth medium is 14-70 mg-N/L (1-4 mM) and the concentration of selenate in the second medium is 100-200 mg/L (0.7-1.4 mM).

### BRIEF DESCRIPTION OF THE DRAWINGS

FIG. 1 illustrates, in accordance with certain embodiments, a schematic of an exemplary bench-scale form of the selenite removal bioreactor system.

FIG. 2 depicts, in accordance with certain embodiments, a profile of Se species after treatment versus increased H₂-supply pressure. The grey frame with dashed line indicates the optimal range of H₂ supply that leads to maximum selenate reduction and Se⁰ production but minimal Se(-II) production.

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

The EPA and North American Metals Council Selenium Workgroup identified biological-reduction technologies as the most reliable and cost-effective alternatives for Se control in aqueous waste streams. Bacteria are able to grow through selenium respiration by reducing selenate to selenite and then selenite to elemental Se (Se⁰), which is an insoluble solid that is immobilized within the biomass matrix:

SeO₄²⁻+2H⁺+2e⁻→SeO₃²⁻+H₂O (1)

SeO₃²⁻+6H⁺+4e⁻→Se⁰+3H₂O (2)

Similar to other biogenic metal(loid) particles, the biogenic Se⁰ can be separated from biomass and recovered through centrifugation or pyrolysis. The recovered Se⁰ can either be disposed of safely or reused as a renewable resource. For example, Se⁰ is a valuable feedstock for the electronics industry, particularly when the Se⁰ is in the nanoparticle form. Thus, environmental protection by selenate bioreduction and removal also can generate an economic benefit.

Selenate bioreduction requires that an electron donor be delivered to the microorganisms. Proven electron donors include a variety of organic compounds (such as acetate, lactate, ethanol, and even methane) and reduced inorganic donors, particularly H₂ gas.

The occurrence of undesired Se species, namely selenide and organic-Se, has been observed during biological selenate treatment. The oxidation state of Se in these undesired selenium species is Se(-II), the most reduced form of Se. Thus, the selenium was over-reduced to form products with distinctly greater toxicity than selenate. At pH<6, the dissolved selenide anion becomes volatile hydrogen selenide (H₂Se), the most acutely toxic selenium species. At higher pH, the selenide anion can precipitate with metallic cations (e.g. Fe²⁺, Cd²⁺, and Zn²⁺) to form insoluble metal selenides (MeₓSe_{y}) that associate with biomass matrix.

Three fundamentally distinct metabolic pathways can lead to biological reduction of more oxidized Se to Se(-II). The first is assimilatory reduction selenate for synthesis in the forms of Se(-II)-containing enzymes, amino acids [selenocysteine (Sec) and selenomethionine (SeMet)], and seleno-proteins. Although they account for only ˜1% of the total utilized Se and are stably associated within bacterial cell matrices, these organic-Se species are released in the treatment effluent as part of detached biomass or as soluble forms from dead and lysed biomass. The second is dissimilatory respiration of selenate to selenide anion (Se²⁻) for energy gain:

SeO₄²⁻+8H⁺+8e⁻→Se²⁻+4H₂O (3)

Complete reduction to Se²⁻ can occur with strongly reducing conditions and seems to depend on the bacterial species present. The third metabolic pathway for biological reduction of more oxidized Se to Se(-II) is reduction and methylation of selenate that leads to the formation of volatile dimethyl selenide [(CH₃)₂Se] and dimethyl diselenide [(CH₃)₂Se₂]. This is a detoxification mechanism for the bacteria, but it has negative impacts on ecosystems and human health due to the methylated selenate being released into the environment.

A variety of biological treatment processes have been attempted for biological reduction and removal of Se from water: constructed wetlands, membrane bioreactors (MBRs), biologically active filters (BAFs), upflow anaerobic sludge blankets (UASBs), and fluidized bed biofilm reactors (FBBRs). These processes have not succeeded in maximizing the removal of Se oxyanions while minimizing the release of undesired reduced Se.

The pressing need is for a reliable method that reduces selenate to Se⁰ , avoids further reduction to Se(-II) species, and recovers the Se⁰ as valuable nanoparticles. The described system and methods address the aforementioned problems with selenate bioreduction. Over-reduction of selenate to Se(-II) is most prevalent when the electron donor is over-dosed. It has proven impossible to control the supply rate of the electron donor when it is supplied as an organic compound or by bubbling H₂. The described bioreactor systems and methods maximize selenate reduction to Se⁰ while minimizing selenide or organic-Se production by focusing on precise control of the delivery capacity of the inorganic electron donor, H₂ gas. The described systems and methods enable precise and on-demand H₂ supply based on the H₂ pressure to the membranes by using bubbleless H₂ from a gas-transfer membrane directly to a biofilm on the outside surface of the membrane. Using bubbleless H₂ delivery, the described systems overcome the problems of under- or over-reduction, which are inherent in other approaches and lead to toxic Se species in the effluent.

In one aspect, the disclosure relates to a system for removing and harvesting Se contaminants from a fluid. The fluid comprises a Se contaminant, for example, selenate and selenite. In one embodiment, the system comprises a biofilm anchored to a nonporous hollow-fiber membrane (for example, a nonporous polymeric hollow-fiber membrane) and a hydrogen gas (H₂) source. As such, the described system is a membrane biofilm reactor. The nonporous hollow-fiber membrane allows H₂ to diffuse through the walls in a bubbleless form. The biofilm comprises H₂-utilizing, autotrophic bacteria (also referred to herein as “hydrogenoautotrophic bacteria”) and selenate-reducing bacteria. The hydrogenoautotrophic bacteria utilize H₂ as their electron donor and CO₂/bicarbonate as their carbon source. Compared to heterotrophic bacteria growing on organic electron donors and carbon sources, hydrogenoautotrophic bacteria produce only about 30% of the biomass when reducing the same amount of selenate to Se⁰ (Rittmann and McCarty, 2001). As a consequence, 70% less Se(-II) is produced in the biofilm compared to the heterotrophic process, in which the problem of Se(-II) species has been documented. In some aspects, the hydrogenoautotrophic bacteria also are selenate-reducing bacteria. In particular embodiments, the biofilm comprises bacteria that reduce selenate and nitrate. In other words, the biofilm comprises selenate-reducing and nitrate-reducing bacteria.

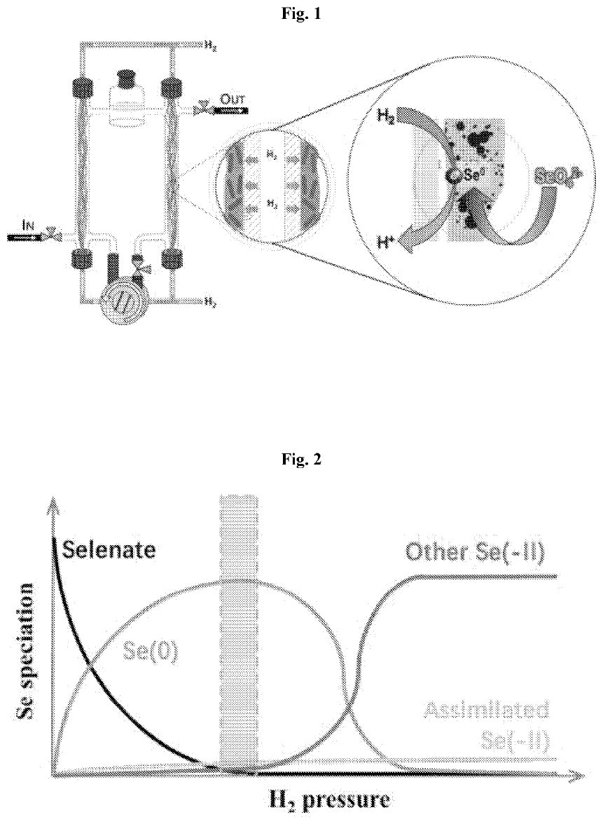

H₂, being an inorganic electron donor to autotrophic bacteria, has inherent advantages over organic electron donors. H₂ is low-cost, nontoxic, and leaves no residual source of electrons in the effluent. H₂ is delivered to the lumen of the nonporous hollow-fiber membrane via the hydrogen gas source at a carefully controlled pressure so that H₂ diffuses through the walls in a bubbleless form. In some aspects, the biofilm is anchored to the outer surface of the hollow-fiber membrane. In other words, H₂ is delivered directly to a self-forming biofilm anchored to the membrane's outer surfaces. This system is illustrated in FIG. 1. Because the final products of dissimilatory selenate reduction are determined by electron-donor delivery, this invention is superior in that it accurately supplies the proper amount of H₂ to maximize production of Se⁰ without surplus H₂ to induce further reduction to Se(-II) species (FIG. 1). By using H₂ as the electron donor and inorganic carbon as the carbon source, the system minimizes the production of biomass, which disposed of and contains undesired organic-Se.

In some embodiments, the hydrogen gas source comprises a gas tank or a hydrogen-gas generator comprising pure H₂ gas and a gas pressure regulator. The gas pressure regulator regulates the flow of H₂ gas from the gas tank or hydrogen-gas generator to the membrane. For example, H₂ gas is delivered into the hollow-fiber such that the H₂ gas is diffused to the biofilm through the membrane. The microorganisms of the biofilm utilize H₂ gas as the electron donor to reduce the selenate or selenite. The reduced Se contaminant is captured in the biofilm as Se⁰ (FIG. 1). In preferred embodiments, the gas regulator ensures the delivery of enough H₂ for complete reduction of selenate (and/or selenite) to Se⁰, but not beyond Se⁰. The H₂-delivery capacity also needs to be adjusted for the H₂ supply needed to co-reduce other electron acceptors, with nitrate being the most likely one in selenate-reduction situations. The H₂ partial pressures required for complete nitrate/nitrite reduction to N₂ gas are equation (4) and equation (5), respectively. The H₂ partial pressures required for complete selenate/selenite reduction to Se⁰ are equation (6) and equation (7), respectively. Equation (8) and equation (9) show the H₂ partial pressures needed to supply enough H₂ for reducing selenate/selenite to Se(-II), respectively; it is greater than for reduction to Se⁰.

$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} \qquad ( 4 )$$

$${P}_{{NO}_{2}^{-} \to {N}_{2}} = 4.7 \times {10}^{- 11 \frac{{C}_{{NO}_{2}^{-} - N}^{in} \cdot Q \cdot {D}_{m} \cdot {d}_{m} \cdot {z}_{m}}{A ( {d}_{m} - {z}_{m} )}} \qquad ( 5 )$$

$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} \qquad ( 6 )$$

$${P}_{{SeO}_{3}^{2 -} \to Se ( 0 )} = 6.1 \times {10}^{- 12 \frac{{C}_{{SeO}_{3}^{2 -}}^{in} \cdot Q \cdot {D}_{m} \cdot {d}_{m} \cdot {z}_{m}}{A ( {d}_{m} - {z}_{m} )}} \qquad ( 7 )$$

$${P}_{{SeO}_{4}^{2 -} \to Se ( - II )} = 1.2 \times {10}^{- 11 \frac{{C}_{{SeO}_{3}^{2 -}}^{in} \cdot Q \cdot {D}_{m} \cdot {d}_{m} \cdot {z}_{m}}{A ( {d}_{m} - {z}_{m} )}} \qquad ( 8 )$$

$${P}_{{SeO}_{3}^{2 -} \to Se ( - II )} = 9.1 \times {10}^{- 12 \frac{{C}_{{SeO}_{3}^{2 -}}^{in} \cdot Q \cdot {D}_{m} \cdot {d}_{m} \cdot {z}_{m}}{A ( {d}_{m} - {z}_{m} )}} \qquad ( 9 )$$

In the six equations, P is the gauge H₂ pressure in the hollow-fiber lumen (psig); Cⁱⁿ is the influent concentration of selenate (mg/L); Q is the flow rate (L/min); A is the total fiber surface area (m²); Dₘ is H₂-diffusion coefficient in the membrane (m²/d); dₘ is hollow-fiber outer diameter (μm), and zₘ is membrane thickness (μm). The H₂ pressure inside the lumen must be kept close to the pressure for reduction to Se⁰, and it must not approach the pressure for reduction to Se(-II).

Table 1 presents typical ranges of selenate/selenite and nitrate/nitrite and the desired ranges of H₂ pressure.

TABLE 1
Operating parameters

| Operating parameter | Unit | Value |
|---|---|---|
| Selenate/selenite | Mg—Se/L | 0.1-100^{α} |
| Nitrate/nitrite | Mg—N/L | 14-70^{α} |
| pH | — | 5.5-8.5 |
| Se surface loading | g/m²/d | 0.01-100 |
| N surface loading | g/m²/d | 0.01-200 |
| Temperature | ° C. | 10-40 |
| H₂ type | — | 10-100% H₂ balanced |
| | | by CO₂ and/or N₂ |
| H₂ partial pressure | psig | 2-30 |

^{α}The nitrate and selenate concentrations may be further altered as needed

FIG. 2 illustrates the strategy for maximizing selenate reduction while minimizing production of over-reduced Se(-II). When H₂ is over-supplied (right side of the figure), selenate is completely removed, but significant portions of the undesired Se(-II) species are released to the effluent. As the H₂ supply is decreased (moving left in the figure), less Se(-II) is released. Moving too far to the left causes an insufficient H₂ supply, which leads to incomplete removal of selenate, another undesired outcome. The optimal H₂ supply pressure for maximum selenate removal and Se⁰ formation occurs in the grey-highlighted region.

In another embodiment, the system comprises a nonporous polymeric hollow-fiber membrane; an inoculant comprising a biofilm-forming population of microorganisms; and a hydrogen gas source. The biofilm-forming population of microorganisms comprises with hydrogenoautotrophic bacteria. In particular aspects, the with hydrogenoautotrophic bacteria comprise nitrate-reducing bacteria and selenate-reducing bacteria.

In some embodiments, the nonporous hollow-fiber membrane comprises polypropylene fibers and has a permeability of 1.8×10⁷ m³ H₂ m membrane thickness/m² hollow-fiber surface area·d·bar at standard temperature and pressure. In some embodiments, the outer diameter of the hollow-fiber membrane is about 200 μm; the inner diameter of the hollow-fiber membrane is about 100-110 μm; and the wall thickness of the hollow-fiber membrane is about 50-55 μm.

The system may further comprise a pump. The pump recirculates the fluid through the system. In preferred embodiments, the pump can recirculate the fluid at a rate of 150 mL/min. The system may further comprise tubing, for example PVC tubing. For bench-scale applications, the tubing is capable of providing an influent feed rate within the range of 0.03-3.00 mL/min.

In some implementations, the system further comprises a means of harvesting Se⁰ from the biofilm. In some aspects, the means of harvesting Se⁰ from the biofilm harvests the biofilm (the biomass of the system). The Se⁰ is harvested by separation from the biomass.

In some embodiments, the system further comprises at least one growth medium. The growth medium stimulates sufficient microbial growth to establish and/or maintain the biofilm. Accordingly, the growth medium stimulates the establishment of nitrate-reducing bacteria and selection for selenate-reducing bacteria. In some aspects, the system comprises a first growth medium and a second growth medium. The first growth medium comprises nitrate as the sole electron acceptor, and it is used until a robust biofilm is established. In certain implementations, the first growth medium comprises 14-70 mg-N/L (1-4 mM) or is synthetic wastewater. In particular implementations, the first growth medium is used in the system for at least three weeks, for example, four weeks, 30 days, or 31 days. Once the biofilm is established, the first growth medium is replaced with the second growth medium, which comprises selenate to enrich the biofilm for selenium-reducing bacteria.

Accordingly, the disclosure also relates to methods of establishing a biofilm in a bioreactor that respires selenate and/or selenite to Se⁰ without producing selenide or organic-Se. The method comprises providing an aqueous system comprising a nonporous hollow-fiber membrane; inoculating the nonporous hollow-fiber membrane with hydrogenoautotrophic bacteria; contacting the aqueous system with hydrogen gas (H₂); providing the inoculated aqueous system with a first growth medium comprising nitrate to establish a biofilm on the nonporous hollow-fiber membrane; and providing the inoculated aqueous system with a second growth medium comprising selenium contaminants to enrich the biofilm for selenium-reducing bacteria. In particular embodiments, the step of inoculating the nonporous hollow-fiber membrane with hydrogenoautotrophic bacteria comprises providing the aqueous system with biomass from a wastewater treatment plant, sediment from a natural body of water (for example, lake, river, or wetland) or with water from a wastewater treatment plant or a natural body of water.

To establish a biofilm that is capable of reducing selenium contaminants to Se⁰ without producing Se(-II) and capturing Se⁰ , the inoculated aqueous system is cultured with the first growth medium comprising nitrate for at least three weeks. Preferably, the first growth medium contains 14-70 mg-N/L (1-4 mM) nitrate as the sole electron acceptor. Accordingly, the first growth medium is provided to the inoculated aqueous system for three to four weeks or one month in some implementations. In some implementations, the first growth medium is provided to the inoculated aqueous system for longer periods until a robust biofilm is established. The biofilm is then enriched for selenium-reducing bacteria by then culturing the inoculated aqueous system with a second growth medium that contains selenate. In certain implementations, the second growth medium contains 100-200 mg/L (0.7-1.4 mM) selenate. In some aspects, the second growth medium is provided to the inoculated aqueous system for at least three weeks, for example, three weeks, four weeks, 30 days, or 31 days. In particular implementations, the method further comprises routinely collecting liquid samples from the aqueous system to monitor nitrate, selenate, selenite, elemental selenium, selenide, and organic-Se in the effluent of the system (see FIG. 1). It is preferable that the effluent is monitored routinely for the first three weeks of culturing with the first growth medium and the first three weeks of culturing with the second growth medium. In certain implementations, the period of culturing the inoculated aqueous system with the first growth medium and the second growth medium can take as long as 160 days or six months.

In particular implementations, the growth medium (either the first or the second) is provided to the inoculated aqueous system at a flow rate of between 0.03-3.0 mL/min, preferably between 0.03-1.0 mL/min or between 0.03-0.10 mL/min. For example, the growth medium is provided to the inoculated aqueous system at a flow rate of 0.03±0.01 mL/min, 0.04±0.01 mL/min, 0.05±0.01 mL/min, 0.06±0.01 mL/min, 0.07±0.01 mL/min, 0.08±0.01 mL/min, 0.09±0.01 mL/min, or 0.10±0.01 mL/min. In some aspects, the growth medium is provided to the inoculated aqueous system at a flow rate of less than 0.1 mL/min or at a hydraulic retention time (HRT) of greater than 12 hours.

The theoretical pressure of H₂ that should be provided to the aqueous system is determined by combining the partial pressures of H₂ calculated from equation (4) and equation (6). The actual H₂ flux is within±10% of the theoretical flux. Accordingly, the pressure of H₂ provided to the aqueous system is range of±10% of the theoretical pressure of H₂ determined by combining the partial pressures of H₂ calculated from equation (4) and equation (6). For example, for a 60-mL reactor that contains 100 cm² of polypropylene nonporous hollow-fiber membranes (Dₘ=1.4×10⁻⁷ m²/d for H₂) with an outer diameter of 200 μm and thickness of 55 μm, the partial pressure of H₂ provided to the reactor is between 2 and 30 psig, preferably between 17.7 to 21.7 psig. In some aspects, the pressure of H₂ provided to the aqueous system during the first stage of establishing the biofilm that is capable of reducing selenium contaminants to Se⁰ without producing selenide or organic-Se and capturing Se⁰ (where the inoculated aqueous system is cultured with the first growth medium) is between ±10% of the pressure calculated from equation (4). In some aspects, the pressure of H₂ provided to the aqueous system during the second stage of establishing the biofilm that is capable of reducing selenium contaminants to Se⁰ without producing selenide or organic-Se and capturing Se⁰ (where the inoculated aqueous system is cultured with the second growth medium) is between ±10% of the pressure calculated from equation (6).

In some implementations of the methods of the invention, the nonporous hollow-fiber membrane comprises hollow-fibers having an outer diameter of 200-300 μm, preferably 200-280 μm, for example 200 μm or 280 μm. The inner diameter of the hollow-fibers of the hollow-fiber membrane may be 100-110 μm. In some aspects, the cross sectional area of the hollow-fibers of the hollow-fiber membrane is 31,000-66,000 μm², for example 31,000-36,000 μm², 36,000-41,000 μm², 41,000-46,000 μm², 46,000-51,000 μm², 51,000-56,000 μm², 56,000-61,000 μm², 61,000-66,000 μm², or preferably 31,000-32,000 μm², 61,000-62,000 μm², 61,000-61,500 μm², 61,500-62,000 μm², or more preferably 31,400 μm² or 61,544 μm². The wall thickness of the hollow-fibers of the hollow-fiber membrane may be 50-70 μm, for example between 50-55 μm, 55-60 μm, 60-65 μm, 65-70 μm, or preferably between 55 μm, 55 μm, or 67 μm. In some embodiments, the hollow-fiber membrane is made of composite material, polyester material, or polypropylene material. For example, the nonporous hollow-fiber membrane comprises composite hollow-fiber, a polyester hollow-fiber, or a polypropylene hollow-fiber.

The methods described herein are also directed to a method for removing selenium contaminants from a fluid, such as wastewater, and to a method for harvest elemental selenium, Se⁰ , from the fluid. These methods comprise first establishing a biofilm that is capable of reducing selenium contaminants to Se⁰ without producing Se(-II) (including selenide or organic-Se) and capturing Se⁰ to establish a bioreactor that reduce selenium contaminants to Se⁰ and then providing to the bioreactor the fluid that contains selenium contaminants. The biofilm reduces the selenium contaminants to Se⁰ , which is a solid captured in the biofilm. Accordingly, Se⁰ may be harvested by harvesting the biomass, which comprises the biofilm, by methods well established in the prior art. The solid Se⁰ may then be separated from the harvested biomass.

### Illustrative, Non-Limiting Example in Accordance with Certain Embodiments

The present invention is further illustrated by the following example that should not be construed as limiting. The contents of all references, patents, and published patent applications cited throughout this application, as well as the Figures, are incorporated herein by reference in their entirety for all purposes.

### 60-mL Reactor for Production Elemental Selenium.

A 60-mL bioreactor system containing a biofilm established to reduce selenium and nitrate comprises 100 cm² of polypropylene nonporous hollow-fiber membranes (Dₘ=1.4×10⁻⁷ m₂/d for H₂) with an outer diameter of 200 μm and thickness of 55 μm. The reactor is continuously fed with a wastewater containing 150 mg/L (˜1 mM) selenate at a surface loading of 1 g-Se/m²/day and 40 mg-N/L (˜3 mM) nitrate at a surface loading of 0.5 g-N/m²/day. Using equation (4) for calculation, the H₂ pressure needed to completely reduce the nitrate to N₂ gas is 13.7 psig. Using equation (6) for calculation, the H₂ pressure needed to completely reduce the selenate to elemental selenium is 6.0 psig. Using equation (8) for calculation, the H₂ pressure needed to completely reduce the selenate to Se(-II) (selenide or organic-Se) is 8.0 psig. The theoretically optimal H₂ pressure that allows complete conversion of nitrate to N₂ and selenate to elemental selenium with minimal production of selenide or organic-Se is 19.7 psig (13.7 psig+6.0 psig). The actual H₂ flux is within ±10% of the theoretic flux. Thus, the estimation suggests a range of desired H₂ pressure from 17.7 to 21.7 psig.

1. A method of establishing a biofilm in a bioreactor to respire selenate and/or selenite to Se⁰ , the method comprising:
providing an aqueous system comprising a nonporous hollow-fiber membrane;
inoculating the nonporous hollow-fiber membrane with hydrogenoautotrophic bacteria;
contacting the aqueous system with hydrogen gas (H₂), wherein the partial pressure of H₂ provided to the aqueous system is±10% of the theoretical pressure of H₂ determined by combining the partial pressures of H₂ calculated from equations (4) and (6):
$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} \qquad ( 4 )$$
$$and$$
$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} \qquad ( 6 )$$
providing the inoculated aqueous system with a first growth medium comprising nitrate to establish a biofilm on the nonporous hollow-fiber membrane; and
providing the inoculated aqueous system with a second growth medium comprising selenium contaminants to enrich the biofilm for selenium-reducing bacteria.

2. The method of claim 1, wherein the biofilm respires selenate and/or selenite to Se⁰ without producing selenide or organic-Se.

3. The method of claim 1, wherein the inoculated aqueous system is cultured with the first growth medium for at least three weeks and cultured with the second growth medium for at least three weeks.

4. The method of claim 1, wherein the second growth medium lacks nitrate.

5. The method of claim 1, wherein the first growth medium contains only nitrate as an electron acceptor.

6. The method of claim 1, wherein the concentration of nitrate in the first growth medium is 14-70 mg-N/L (1-4 mM) and the concentration of selenate in the second medium is 100-200 mg/L (0.7-1.4 mM).

7. A method for removing selenium contaminants from a fluid, the method comprising:
providing an aqueous system comprising a nonporous hollow-fiber membrane;
inoculating the nonporous hollow-fiber membrane with hydrogenoautotrophic bacteria;
contacting the aqueous system with hydrogen gas (H₂), wherein the partial pressure of H₂ provided to the aqueous system is ±10% of the theoretical pressure of H₂ determined by combining the partial pressures of H₂ calculated from equations:
$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} and , \qquad ( 4 )$$
$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} ; \qquad ( 6 )$$
providing the inoculated aqueous system with a first growth medium comprising nitrate to establish a biofilm on the nonporous hollow-fiber membrane;
providing the inoculated aqueous system with a second growth medium comprising selenium contaminants to enrich the biofilm for selenium-reducing bacteria, whereby a bioreactor for reducing selenate is produced; and
contacting the bioreactor for reducing selenate with fluid containing selenium contaminant, whereby the biofilm enriched for selenium-reducing bacteria reduces selenium contaminate to Se⁰ and captures Se⁰.

8. The method of claim 7, further comprising harvesting the biomass in the bioreactor to harvest Se⁰ generated by the bioreactor.

9. The method of claim 7, wherein the biofilm respires selenate and/or selenite to Se⁰ without producing selenide or organic-Se.

10. The method of claim 7, wherein the inoculated aqueous system is cultured with the first growth medium for at least three weeks and cultured with the second growth medium for at least three weeks.

11. The method of claim 7, wherein the second growth medium lacks nitrate.

12. The method of claim 7, wherein the first growth medium contains only nitrate as an electron donor.

13. The method of claim 7, wherein the first growth medium contains only nitrate.

14. The method of claim 7, wherein the concentration of nitrate in the first growth medium is 14-70 mg-N/L (1-4 mM) and the concentration of selenate in the second medium is 100-200 mg/L (0.7-1.4 mM).

15. A system for removing selenium contaminants and harvesting elemental selenium (Se⁰) from a fluid, the system comprising:
a nonporous hollow-fiber membrane;
an inoculant comprising hydrogenoautotrophic bacteria; and
a hydrogen gas source.

16. (canceled)

17. The system of claim 15, wherein the hydrogen gas source provides H₂ at partial pressure of ±10% of the theoretical pressure of H₂ determined by combining the partial pressures of H₂ calculated from equation:
$${P}_{{NO}_{3}^{-} \to {N}_{2}} = 7.8 \times {10}^{- 11 \frac{{C}_{{NO}_{3}^{-} - N}^{in} \cdot Q \cdot {D}_{m} \cdot {d}_{m} \cdot {z}_{m}}{A ( {d}_{m} - {z}_{m} )}} and , \qquad ( 4 )$$
$${P}_{{SeO}_{4}^{2 -} \to Se ( 0 )} = 9.7 \times {10}^{- 12 \frac{{C}_{{SeO}_{4}^{2 -}}^{in} \cdot Q \cdot {D}_{m} \cdot {d}_{m} \cdot {z}_{m}}{A ( {d}_{m} - {z}_{m} )}} . \qquad ( 6 )$$

18. The system of claim 15, further comprising a growth medium, the growth medium comprising selenate.

19. (canceled)

20. The system of claim 15, further comprising a first growth medium and a second growth medium, wherein the first growth medium comprises nitrate and the second growth medium comprises selenate.

21. The system of claim 20, wherein the second growth medium lacks nitrate.

22. The system of claim 20, wherein the first growth medium contains only nitrate as an electron donor.

23. (canceled)
